(19) United States
(12) Patent Application Publication
(10) Pub. No.: US 2006/0293753 A1
(43) Pub. Date: Dec. 28, 2006

(54) CORRECTIVE ARTIFICIAL DISC

(75) Inventor: Jeffery Thramann, Longmont, CO (US)

Correspondence Address: HOLLAND & HART, LLP, P.O BOX 8749, DENVER, CO, 80201, US

(73) Assignee: Lanx, LLC, Boulder, CO (US)

(21) Appl. No.: 11/421,536

(22) Filed: Jun. 1, 2006

Related U.S. Application Data
(63) Continuation-in-part of application No. 10/641,530, filed on Aug. 14, 2003, now Pat. No. 7,101,400.
(60) Provisional application No. 60/404,481, filed on Aug. 19, 2002.

(51) Int. Cl. A61F 2/44

(52) U.S. Cl. 623/17.13; 606/78; 623/17.11

(57) ABSTRACT

An artificial disc is provided having a rigid pre-formed phase. The disc is implanted in an elastic phase and activated to the pre-formed phase. The pre-formed shape provides corrective forces to stabilize or correct spinal abnormalities.

### RELATED APPLICATIONS

This application is a continuation in part application of U.S. patent application Ser. No. 10/641,530, titled SHPAED MEMORY ARTIFICIAL DISC AND METHODS OF ENGRAFTING THE SAME, filed Aug. 14, 2003, which claims the benefit of U.S. Provisional Patent Application 60/404,481, filed Aug. 19, 2002, titled SHAPE MEMORY ARTIFICIAL DISC.

### FIELD OF THE INVENTION

The present invention relates to artificial intervertebral discs and, more particularly, artificial intervertebral discs constructed from shaped memory alloys that can be used to correct spinal abnormalities.

### BACKGROUND OF THE INVENTION

The vertebrae of the human spine are arranged in a column with one vertebra on top of the next. Between adjacent vertebrae exists an intervertebral disc that transmits force between adjacent vertebrae and provides a cushion between the adjacent vertebrae.

The spine can suffer from many degenerations, diseases, and deformities that can cause back pain (hereinafter “spinal abnormality”). Some spinal abnormalities impinges upon neurological structures or is determined to be a pain generator. Surgeons attempt to treat these pain generators using therapy, medications, and in some cases, surgery. For example, conventionally treat of spinal pain is to insert a bone graft or other device in the space vacated by the diseased disc. The adjacent vertebrae are then immobilized relative to one another with stabilization hardware. Eventually, the vertebrae grow into one solid piece of bone, which relieves the pain.

While fusing the vertebrae into one solid piece of bone is the conventional practice, fusing adjacent vertebrae into a single bone mass is a less than ideal solution. In particular, fusing two or more vertebrae into a single bone mass causes additional stress on the remaining vertebrae and discs accelerating any potential degeneration. Moreover, the fused bone mass may lead to decreased motion and flexibility in the spine. The decreased motion and/or flexibility is exacerbated when three or more vertebrae are fused.

In order to avoid fusing two or more vertebrae into a single bone mass, prosthetic devices have been developed that attempt to mimic the intervertebral disc, both size and function. The prosthetic device is implanted into the intervertebral space to replace the diseased disc. U.S. Pat. No. 5,458,642, titled SYNTHETIC INTERVERTEBRAL DISC, issued Oct. 17, 1995, to Beer et al. discloses one such prosthetic device. The Beer et al. device includes a plurality of coiled springs interspersed between two endplates. The springs of the Beer et al. device attempt to approximate the function of the replaced intervertebral disc. The Beer et al. device is less than satisfactory because the coiled springs can be damaged and lose their elasticity over time. Further, the coiled springs provide limited shock absorption requiring the use of a compressible pouch of biocompatible material to provide additional shock absorption. Moreover, adjacent vertebrae need significant separation to allow for insertion of the prosthetic device potentially causing trauma to the surrounding structures.

U.S. Pat. No. 5,676,702, titled ELASTIC DISC PROSTHESIS, issued Oct. 14, 1997, to Ratron, provides another device that attempts to mimic the replaced intervertebral disc. The Ratron device includes the same endplates separated by an elastic post and elastically deformable partitions. The Ratron device is relatively impractical, however, because the placement of the elastic post and the elastically deformable partitions is difficult and varies on a case-by-case basis. Thus, manufacturing the device prior to surgical implantation is difficult. Further, bone or other tissue growth into the intervertebral space can foul the device making it inoperable. Moreover, adjacent vertebrae need significant separation to allow for insertion of the prosthetic device potentially causing trauma to the surrounding structures. Finally, similar to springs, the elastic material may experience plastic deformation causing failure of the prosthesis. Additionally, the elastic material contained in the Ratron device may degrade over time.

U.S. Pat. No. 6,770,094, titled INTERVERTEBRAL DISC PROSTHESIS, issued on Aug. 3, 2004, to Fehling et al., incorporated herein by reference as if set out in full, describes still another intervertebral disc. The '094 patent uses shaped memory alloys to simulate the elastic response of the anatomical disc. Thus, the shaped memory alloy operates in its superelastic or martensite phase. While not proven, theoretically the SMA mimics the elastic response of the disc nucleus and could be a functional equivalent replacement.

However, other abnormalities also may cause back or spinal problems. For example, scoliosis relates to an abnormal curvature of the spine. Correction of scoliosis involves cumbersome braces or relatively invasive surgery to implant a corrective rod. The corrective rod, typically a pair of rods, apply force to the spine to prevent further curvature or provide corrective force to the spinal column to correct the abnormal curvature. Moreover, none of the above mentioned devices provide any mechanism to stabilize or correct the scoliosis.

Thus, it would be desirous to develop an improved device and method to correct spinal abnormalities.

### SUMMARY OF THE INVENTION

To attain the advantages and in accordance with the purpose of the invention, as embodied and broadly described herein, an artificial intervertebral disc is provided. The artificial disc includes at least one superior endplate and at least one inferior endplate. At least one core extends between the at least one superior endplate and the at least one inferior endplate. The core comprises a material movable from a non-corrective shape to a corrective shape, such that transitioning the at least one core from the elastic phase to the inelastic phase applies corrective force to a spine.

The foregoing and other features, utilities and advantages of the invention will be apparent from the following more particular description of a preferred embodiment of the invention as illustrated in the accompanying drawings.

### BRIEF DESCRIPTION OF THE DRAWING

The accompanying drawings, which are incorporated in and constitute a part of this specification, illustrate embodiments of the present invention, and together with the description, serve to explain the principles thereof. Like items in the drawings are referred to using the same numerical reference.

FIG. 1 is a cross-sectional, anterior view of adjacent vertebral bodies with an artificial disc consistent with an embodiment of the present invention;

FIG. 2 is a lateral elevation view of the device of FIG. 1;

FIG. 3 is a cross-sectional, anterior view of adjacent vertebral bodies with an artificial disc consistent with another embodiment of the present invention;

FIG. 4 is a cross-sectional, anterior view of adjacent vertebral bodies with an artificial disc consistent with another embodiment of the present invention; and

FIG. 5 is an elevation view of an artificial disc consistent with yet another embodiment of the present invention shown interlocked;

FIG. 6 is a view of possible shapes for the shaped memory alloy members 124 shown in FIG. 2;

FIG. 7 is an elevation view of the disc of FIG. 5 shown not interlocked;

FIG. 8 is a view of a force applied by a corrective disc consistent with an embodiment of the invention;

FIG. 9 is a view of an unbalanced corrective disc consistent with an embodiment of the invention;

FIG. 10 is a view of a support wall useable with the present invention for disc annulus support; and

FIG. 11 is a view of a view of interlocking portion 506 in an interlocked position.

### DETAILED DESCRIPTION

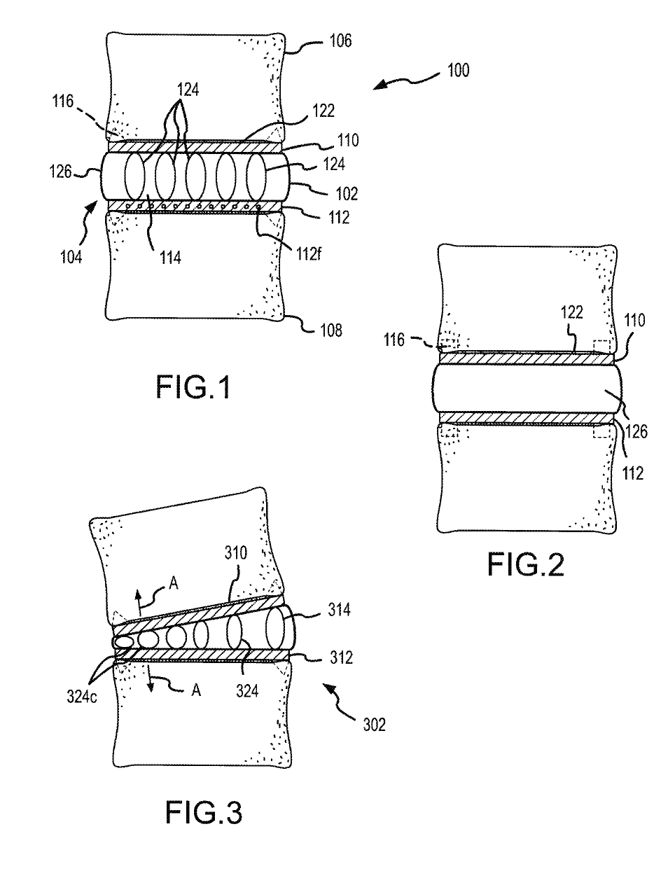

Some embodiments of the present invention are described with reference to FIGS. 1 to 11. FIGS. 1-11 generally show the present invention on an eye level, off the shelf view with fixation spikes in phantom. One of skill in the art, on reading the below disclosure, will recognize that the exact configuration of the present invention will depend, in part, on the anatomy of the patient.

In particular, FIG. 1 shows a cross section of an anterior view of a portion of a spinal column 100 with an artificial disc 102. FIG. 2 shows a lateral, elevation view of spinal column 100 with artificial disc 102. Disc 102 is implanted in an intervertebral space 104 situated between a superior vertebral body 106 and an inferior vertebral body 108. Disc 102 includes a superior endplate 110, an inferior endplate 112, and a core 114. Superior endplate 110 and inferior endplate 112 may be formed of a biocompatible metal including shaped memory alloys, other metallic alloys, PEEK, resorbable, plastic, biologics, or the like. Conventionally, endplates are made from titanium, but any biocompatible material is satisfactory. Moreover, superior endplate 110, inferior endplate 112, or a combination thereof could be constructed with fusion cages 112f or the like, similar to U.S. patent application Ser. No. 11/163,299, filed Oct. 13, 2005, titled ARTIFICIAL DISC WITH ENDPLATES HAVING CAGES TO PROMOTE BONE FUSION, and incorporated herein by reference as if set out in full. Referring to FIG. 1, superior endplate 110 is shown as a conventional endplate while inferior endplate 112 is shown with fusion cage 112f.

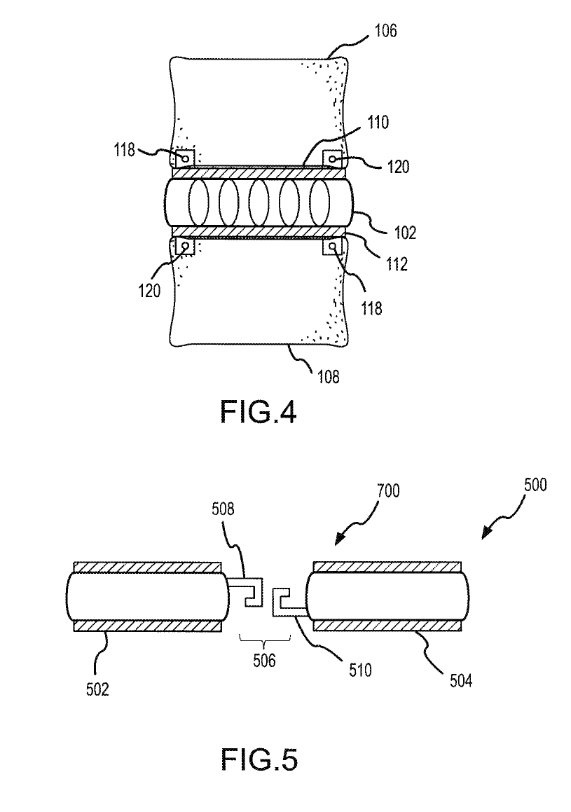

If, for example, superior endplate 110 and inferior endplate 112 are made from shaped memory alloys, the plates could be placed in a compact state (deformed, rolled, coiled, or the like) and activated once placed. Superior endplate 110 is coupled to superior vertebral body 106 using, for example, fixation spikes 116 (shown in phantom on FIGS. 1, 2, and 3), bone growth, fusion, or the like. Fixation spikes could be replaced with screws or other conventional coupling devices. FIG. 4 shows an alternative attachment means where, for example, endplates 110 and 112 have fixation tabs 118 through which screws 120 are inserted to couple endplates 110 and 112 to superior vertebral body 106 and inferior vertebral body 108. Such fixation devices may be comprised of resorbable material to facilitate the fixation.

Superior endplate 110 and inferior endplate 112 are shown with a lattice, mechanical, and/or biochemical coating 122 to enhance bone ingrowth and encourage long-term fixation of the plates to the vertebral bodies. Alternative to coating the plates with bone growth material, the coating could include, for example, ridges, ribs, scars, striations, or the like. Further, a layer of adhesive or tape could be applied to assist in fixation of the plates. Moreover, a resorbable plate or the like may be arranged fixing superior vertebral body 106 and inferior vertebral body 108. Fixing the motion of the two vertebral bodies would facilitate endplates fusing to the bone. Once the endplates fuse to the bone, the resorbable plate or the like would degrade allowing the artificial disc to move.

Core 114 comprises a shaped memory alloy (SMA). SMAs are a group of materials that demonstrate an ability to return to some previously defined shape or size when subjected to the appropriate thermal procedure. Generally, these materials can be plastically deformed at a predefined temperature, and upon exposure to thermal manipulation, will return to the pre-deformation shape. Some SMA material is considered to be two-way shaped memory alloys because they will return to the deformed shape upon proper thermal activation. SMAs include Ag—Cd alloys, Cu—Al—Ni alloys, Cu—Sn alloys, Cu—Zn alloys, Cu—Zn—Si alloys, Cu—Zn—Sn alloys, Cu—Zn—Al alloys, In—Ti alloys, Ni—Al alloys, Ni—Ti alloys, Fe—Pt alloys, Mn—Cu alloys, Fe—Mn—Si alloys, and the like. In other words, these materials have a martensite or super elastic structure and an austenite or rigid structure.

Some artificial discs, such as Fehling et al., are beginning to incorporate SMAs as the elastic material or core 106 of artificial discs. This is in part because in the martensite, super elastic structure, the SMA can absorb forces laterially, medially, and torsionally, similar to the biological disc.

Currently, Ni—Ti alloys (a.k.a. Nitinol) are considered a good SMA for medical applications. Making core 114 out of SMAs provides the ability to implant a compact artificial disc during the procedure requiring less distraction of upper vertebral body 106 and lower vertebral body 108. An activation signal would be provided to cause core 114 to expand to the surgically desired shape. The reduction in distraction reduces the surgical trauma associated with the implant. Many SMAs are thermally activated, however, activation signals may be any number of signals, such as, for example, thermal, electrical, magnetic, radiation, or the like.

However, while SMAs operating in the super elastic or martensite structure may provide treatment for some spine diseases that previously required fusion, the same products fail to provide any corrective or therapeutic treatment for other spinal abnormalities, such as, scoliosis.

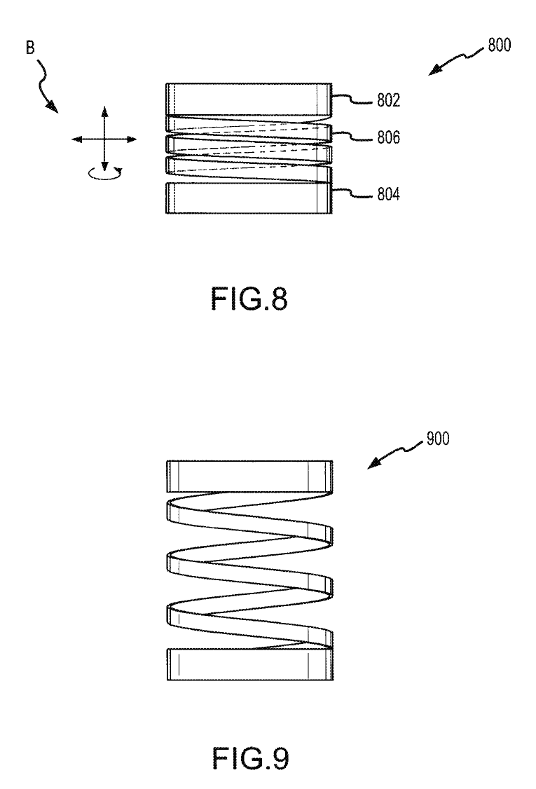

Referring now to FIG. 8, a disc 800 is shown. For simplicity, disc 800 is shown with superior endplate 802, inferior endplate 804, and core 806 comprising a single coil spring of SMA material. Core 806 while shown using an SMA material, may be replaced by polymers, resins, composites, and the like capable of being inserted and activated or malipulated after implantation to a pre-formed shape. SMAs are considered good materials because they can be implanted in the super elastic, martensite phase, and activated (typically by thermal activation) to transition to the austenite phase. Disc 800 would typically be implanted in the elastic phase to facilitate ease of implantation, but the initial implantation shape does not need to be the super elastic phase, but rather a first or non-corrective position. Thus, when activated, the material attempts to return to its pre-formed austenite shape, i.e., a second or corrective position. The pre-formed shape can be designed to apply corrective force in the lateral, medial and torsional directions as shown by arrows B. Thus, core 806 applies a corrective force between superior vertebral body 106 and inferior vertebral body 108. Unlike current artificial discs, disc 800 can be used to apply corrective forces to stabilize an abnormally shaped spinal column, correct the abnormally shaped spinal column, or other malady. Moreover, once the abnormality is corrected, SMAs, for example, can be forced back to the martensite structure to provide super elastic properties and behave as an artificial disc. Operation of a single corrective disc is described, but multiple discs may be necessary to provide proper corrective force.

Activation may occur immediately after implant, but to avoid trauma to the bone and spine, superior endplate 802 and inferior endplate 804 may be provided sufficient time to fuse with the superior and inferior vertebral bodies.

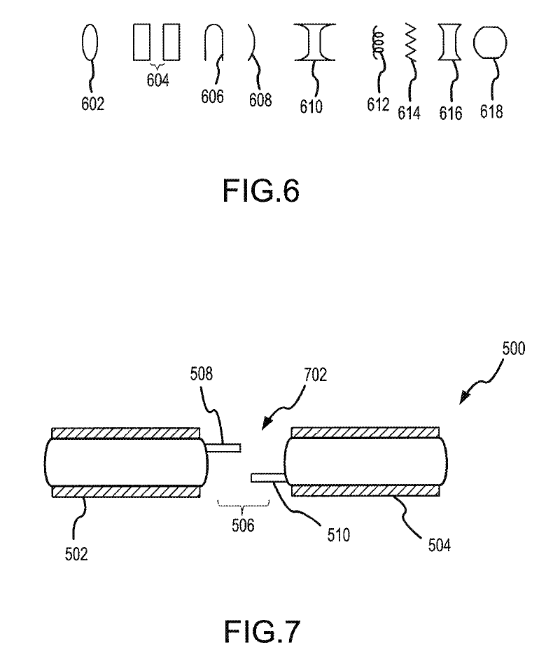

While core 114 could be a solid piece of SMA material, such as core 806 having a single coil spring, it is believed core 114 would function better if core 114 comprises a series of SMA members 124 extending between upper endplate 110 and lower endplate 112. As shown, each SMA member 124 is a loop 602 (shown in FIG. 6) of SMA material to provide some vertical elastic deformation ability. As shown in FIG. 6, SMA members 124, however, could have a number of constructions, including, for example, a series of columns 604, an arc 606 or curved shape 608, a flanged surface(s) 610, coils 806, a zigzag pattern 614, a bi-convex shape 616, a biconcave shape 618, or the like. Further, each member 124 could have different or the same construction and made out of different or the same SMAs. The variation of the shapes and materials would provide surgical control of the forces associated with core 114. In particular, the SMAs could be chosen, shaped, and designed to specifically provide, replicate, or resist axial, rotational, sagital, and coronal forces.

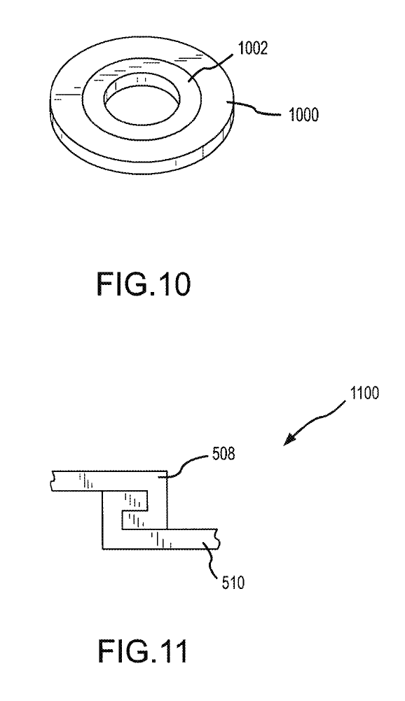

A covering 126 may be deployed around core 114 to prevent tissue, scarring, or bone growth from interfering with disc 102. Covering 126 could be formed of a biocompatible metal, an alloy, or plastic. For example, covering 126 could be a GORTEX® material, but any biocompatible material would function. Alternatively to covering 126, the annulus surrounding the disc could be used. In this case, the surgeon would preserve the majority of the annulus. Alternatively, a repair or support structure could be implanted to the annulus. As shown in FIG. 10, disc annulus 1000 may be augmented by a support wall 1002 could be threaded into the disc annulus area. Support wall 1002 may be used in combination with a scaring material 1004, such as cotton. Scaring material 1004 could be used alone to promote fibrous growth to repair disc annulus 1000 as well. Support wall 1002 may be loaded with pharmaceuticals 1006 as desired.

FIGS. 1, 2, and 8 show disc 102 with a “balanced” core 114, 806. Basically, balanced means that SMAs 124 are approximately identical. Referring specifically to FIG. 3, disc 302 is shown. Disc 302 has an “unbalanced” core 314 where the individual SMAs 324 contained in core 314 are designed with different sizes, radii, and elastic deformation coefficients. FIG. 9 shows a single unbalanced SMA element 900. While SMA element 900 is shown as a coil spring, other elements shapes are possible as otherwise described herein. As implanted, unbalanced disc 302 applies different forces than a balanced disc would deploy. Further, because of the unique feature associated with SMA material, compact SMAs 324c could be implanted in an unexpanded state. Over time, to potentially aid in correcting spinal curvature, thermal activation of SMAs 324c could apply expansion force causing SMAs 324c to become less compact and move disc 302 from unbalanced core 314 to a more balanced core formation. The expansion of SMAs 324c would apply a force represented by Arrows A and would be designed to correct the spines curvature or the like. Alternatively to an unbalanced core, as shown in FIG. 3, multiple discs could be used aligned within the intervertebral space. Each of these multiple discs may be have a balanced core, but the core of the first disc may be different than the core of the second disc, etc., which would approximate the effect of an unbalanced core. Consistently, the individual SMA elements may each be unbalanced as well.

Conventionally, artificial discs are implanted using anterior surgical techniques. FIG. 5 shows that an artificial disc 500. Artificial disc 500 is essentially the same as disc 102 and disc 302. Disc 500 is divided into a first part 502 and a second part 504. Disc 500 has endplates, bone growth material, and a core similar to the above discs, and those pieces will not be re-explained here. An interlocking portion 506 has a first interlocking piece 508 attached to first part 502 and a second interlocking piece 510 attached to second part 504. While shown exploded for convenience in FIG. 5, piece 508 and 510 are shown intertwined in FIG. 11. FIG. 11 shows pieces 508 and 510 in the locked position 1100. FIG. 7 shows disc 500 with first interlocking piece 508 and second interlocking piece 510 in an unlocked position 702. Unlocked position allows piece 508 and 510 to move or slide relative to one another. If, for example, pieces 508 and 510 comprised SMAs, activating pieces 508 and 510 could take them from the unlocked position shown in FIG. 7 to the interlocked position shown in FIG. 11. While interlocking portion 506 is shown as a relatively simple device, one of ordinary skill in the art would recognize other more elaborate interlocking portions could be designed and used and the simple design shown is for convenience and ease of explanation. Interlocking pieces 508 and 510 can be attached to parts 502 and 504 by being a single integrated unit, screwed, glued, taped, adhered, or the like. Interlocking pieces 508 and 510 could be made of SMAs. Activated, interlocking pieces 508 and 510 engage and hold first part 502 and second part 504 together. To install, however, interlocking pieces 508 and 510 would be in a non-activated position allowing first part 502 and second part 504 to move relative to each other. Using two halves, disc 500 can be installed from a posterior procedure. Implanting an artificial disc using a posterior procedure would be a vast improvement over current anterior implanting procedures because of the reduction in surgical trauma. Once installed, activation of interlocking portion 506 would cause the interlocking pieces 508 and 510 to engage.

While FIG. 5 shows installing two halves of an artificial disc and linking the two halves together with interlocking portion 506, it would be possible to implant several artificial disc modules in a side-by-side relation. The side-by-side modules could be linked (similar to FIG. 5) or function independently. Using several smaller modules to mimic the removed disc instead of one larger artificial disc would facilitate implantation of the artificial disc using minimally invasive techniques.

While the invention has been particularly shown and described with reference to an embodiment thereof, it will be understood by those skilled in the art that various other changes in the form and details may be made without departing from the spirit and scope of the invention.

We claim:

1. A method to correct spinal deformities, comprising the steps of:
pre-forming an artificial disc with a corrective shape;
moving the artificial disc from the corrective shape to a non-corrective shape;
implanting the artificial disc in the non-corrective shape into an intervertebral disc space;
moving the artificial disc from the non-corrective shape to the corrective shape, wherein
the artificial disc provides corrective force between a superior and 10 inferior vertebral body.

2. The method according to claim 1, wherein the corrective shape is a rigid austenite phase and the non-corrective shape is an elastic, martensite phase.

3. The method according to claim 1, wherein the step of moving the artificial disc comprises applying thermal activation.

4. The method according to claim 2, wherein the pre-formed rigid shape is an austenite shape and the elastic shape is a martensite shape.

5. The method according to claim 2, wherein the step of moving the artificial disc from the elastic shape to the rigid shape occurs after implantation.

6. The method according to claim 5, further comprising the step of fusing the artificial disc to at least one of a superior vertebral body and an inferior vertebral body.

7. The method according to claim 2, wherein the step of moving the artificial disc from the elastic shape to the rigid shape causes the application of force.

8. The method according to claim 7, wherein the application of force apply force in at least one of the medial, lateral, and torsional direction.

9. The method of claim 2, further comprising the step of subsequently moving the artificial disc from the pre-formed rigid, austenite phase to the elastic, martensite phase.

10. The method of claim 1, wherein the spinal abnormality treated is scoliosis.

11. The method of claim 1, wherein steps of pre-forming an artificial disc with a corrective shape, moving the artificial disc from the corrective shape to a non-corrective shape, implanting the artificial disc in the non-corrective shape into an intervertebral disc space, moving the artificial disc from the non-corrective shape to the corrective shape comprises performing the steps on a plurality of artificial discs.

12. An artificial disc comprising:
at least one superior endplate;
at least one inferior endplate;
at least one core extending between the at least one superior endplate and the at least one inferior endplate;
the at least one core comprising a material movable from an elastic phase to an inelastic phase, the material having a pre-defined shape in at least the inelastic phase, such that transitioning the at least one core from the elastic phase to the inelastic phase applies corrective force to a spine.

13. The artificial disc according to claim 12, wherein the pre-defined shape is designed to apply corrective force between an superior vertebral body and an inferior vertebral body.

14. The artificial disc according to claim 12, wherein the material comprises at least one shaped memory alloy.

15. The artificial disc according to claim 14, wherein the artificial disc is unbalanced and application of corrective force moves the artificial disc to a balanced configuration.

16. The artificial disc according to claim 12, further comprising at least one fusion cage on an endplate selected from the group of endplates consisting of: superior endplate and inferior endplate.

17. The artificial disc according to claim 12, wherein the material comprises a plurality of element.

18. The artificial disc according to claim 17, wherein at least one of the plurality of elements is different from the others.

19. The artificial disc according to claim 12, further comprising a support wall to repair a disc annulus.

20. The artificial disc according to claim 19, further comprising pharmaceuticals contained in the support wall.
